(19) United States
(12) Patent Application Publication
(10) Pub. No.: US 2004/0167554 A1
(43) Pub. Date: Aug. 26, 2004

(54) Methods and devices for reentering a true lumen from a subintimal space

(75) Inventors: John B. Simpson, Woodside, CA (US); Michael H. Rosenthal, Palo Alto, CA (US); Gautama B. Venegas, Pleasanton, CA (US); Darren G. Doud, San Jose, CA (US); John E. Ashley, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Fox Hollow Technologies, Inc., Redwood City, CA (US)

(21) Appl. No.: 10/421,980

(22) Filed: Apr. 22, 2003

Related U.S. Application Data
(63) Continuation-in-part of application No. 10/288,581, filed on Nov. 4, 2002.
Application No. 10/288,581 is a continuation-in-part of application No. 10/027,418, filed on Dec. 19, 2001.
(60) Provisional application No. 60/272,273, filed on Feb. 27, 2001.
(60) Provisional application No. 60/257,704, filed on Dec. 20, 2000.

(51) Int. Cl. A61B 17/22

(52) U.S. Cl. 606/159

(57) ABSTRACT

A device for reentering a true lumen from a subintimal location includes a rotating cutting element and energy emitting element. Energy is emitted to determine the location of the true lumen and the cutting element is exposed to cut an access path to the true lumen. The cutting element may be gradually exposed and the distal portion may be bent or articulated to move the cutting element toward the material to be cut.

### CROSS-REFERENCES TO RELATED APPLICATIONS

The present application is a continuation-in-part of U.S. patent application Ser. No. 10/288,581 (Attorney Docket No. 018489-002540US), which is a continuation-in-part of U.S. patent application Ser. No. 10/027,418, filed Dec. 19, 2001, entitled “Debulking Catheter”, which claims the benefit of Provisional Patent Application Serial No. 60/257,704, filed Dec. 20, 2000, entitled “Debulking Catheter” and Provisional Patent Application Serial No. 60/272,273 filed Feb. 27, 2001, entitled “Debulking Catheter”, the complete disclosures of which are incorporated herein by reference.

### BACKGROUND OF THE INVENTION

The present invention is directed to devices and methods for cutting tissue. In a specific application, the present invention is directed to devices and methods for re-entering the true lumen from a subintimal space such as a dissection plane or so-called “false lumen.”

Guidewires and other interventional devices are used to treat vessels and organs using endovascular approaches. A guidewire is typically guided through blood vessels to the treatment site and the device is then advanced over the guidewire. For example, angioplasty and stenting are generally accomplished by first introducing a guidewire to the desired site and then advancing the angioplasty or stent catheter over the guidewire.

When attempting to advance a guidewire or other interventional device through a highly stenosed region or chronic total occlusion (CTO), the guidewire or device may inadvertently enter into the wall of the vessel to create a sub-intimal space. Once in a sub-intimal space, it can be difficult to re-enter the vessel true lumen. Devices for reentering a vessel true lumen from a subintimal location are described in WO 02/45598 which is hereby incorporated by reference.

### BRIEF SUMMARY OF THE INVENTION

Various aspects of the invention are directed to methods and devices for re-entering a lumen during an endovascular procedure. In one embodiment, the device has a cutter, an opening, and an energy emitter coupled to the cutter. The device is advanced into the subintimal space and energy is then emitted from the energy emitter to locate the true lumen. In one aspect, the energy emitter and cutting element are moved together which exposes the cutting element to cut an access path into the true lumen. In another aspect of the present invention, the device may have a bendable tip which is bent while cutting tissue to create the access path or may be bent to direct the device or a guidewire through the access path.

In another aspect of the present invention, the device has a rotatable cutting element which may be moved from a stored position to a cutting position which exposes over 180 degrees, and even 220 or even 270 degrees of the cutting element relative to the axis of rotation. In another aspect of the invention, the cutter may be gradually exposed as necessary. In still another aspect of the present invention, the body of device may be wider along a portion of the device to urge tissue toward the cutting element. The opening is relatively large and may be open at the distal end and may expose at least part of the cutter at all positions distal to the opening. The open end of the device permits the tissue to naturally move toward the cutter due to the generally open nature of the distal end.

In still another aspect of the present invention, a system for accessing a subintimal space includes a catheter through which the tissue cutting device is advanced. The catheter may be coupled to a fluid source to inject contrast or the like and may also be coupled to a pressure monitor for monitoring pressure to determine when the access path has been created as described in greater detail below.

In a still further aspect of the invention, a method of entering a true lumen from a false lumen during an endovascular procedure is provided. A guidewire is positioned in the subintimal space. A reentry device is then advanced over the guidewire to the target location in the subintimal space. The access path is then created using the reentry device to cut the access path. The same guidewire is then directed through the access path. The reentry device may have two different openings with the first being used during advancement of the reentry device and a second opening through which the guidewire extends when being directed through the access path. The first opening may be configured to direct the guidewire substantially longitudinal while the second opening directs the guidewire at an angle relative to the longitudinal axis.

These and other aspects of the invention will become apparent from the following description, drawings and claims.

### BRIEF DESCRIPTION OF THE DRAWINGS

FIG. 1 shows a view of the system of the present invention.

FIG. 2 shows a guidewire positioned proximate to a total occlusion.

FIG. 3 shows a subintimal space created adjacent a true lumen by the guidewire.

FIG. 4 shows a reentry device of the present invention advanced over the guidewire to the subintimal space.

FIG. 5 shows a guidewire positioned in the true lumen.

FIG. 6 shows the reentry device with the cutting element in a stored position.

FIG. 7 shows the reentry device with the cutting element in a cutting position.

FIG. 8 is a side view of the reentry device of FIG. 7.

FIG. 9 shows another reentry device with the cutting element in a stored position.

FIG. 10 shows the reentry device of FIG. 9 with the cutting element in a cutting position and the distal portion bent.

FIG. 11 shows the reentry device of FIGS. 9 and 10 with the cutting element advanced to another cutting position which exposes even more of the cutting element and also bends the distal tip further.

FIG. 12 shows another reentry device which has a bendable distal portion.

FIG. 13 shows the reentry device of FIG. 12 with the distal portion bent.

FIG. 14 shows still another reentry device with a cutting element which may be tilted.

FIG. 15 shows the reentry device of FIG. 14 with the cutting element tilted to expose more of the cutting element and to move the cutting element through the opening in the body of the device.

FIG. 16 shows the reentry device of FIG. 6 having a junction leading to two separate guidewire outlets with the guidewire positioned in the first outlet during advancement of the device over the guidewire.

FIG. 17 shows the reentry device of FIG. 16 with the guidewire extending through the second outlet for directing the guidewire into the true lumen.

FIG. 18 shows a catheter having a lumen for receiving a guidewire and another lumen which receives the reentry device.

FIG. 19 shows another catheter having a single lumen through which the guidewire and reentry device pass.

### DETAILED DESCRIPTION OF THE INVENTION

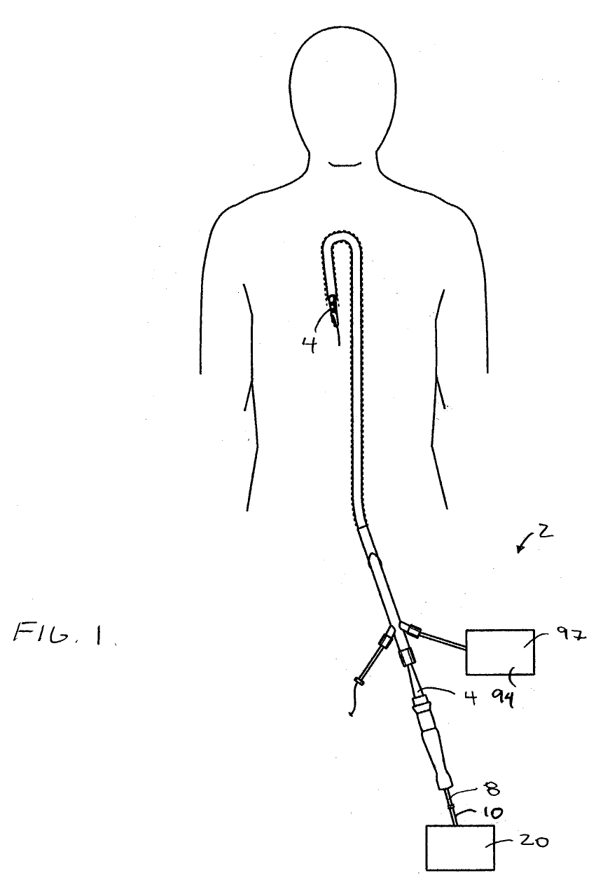

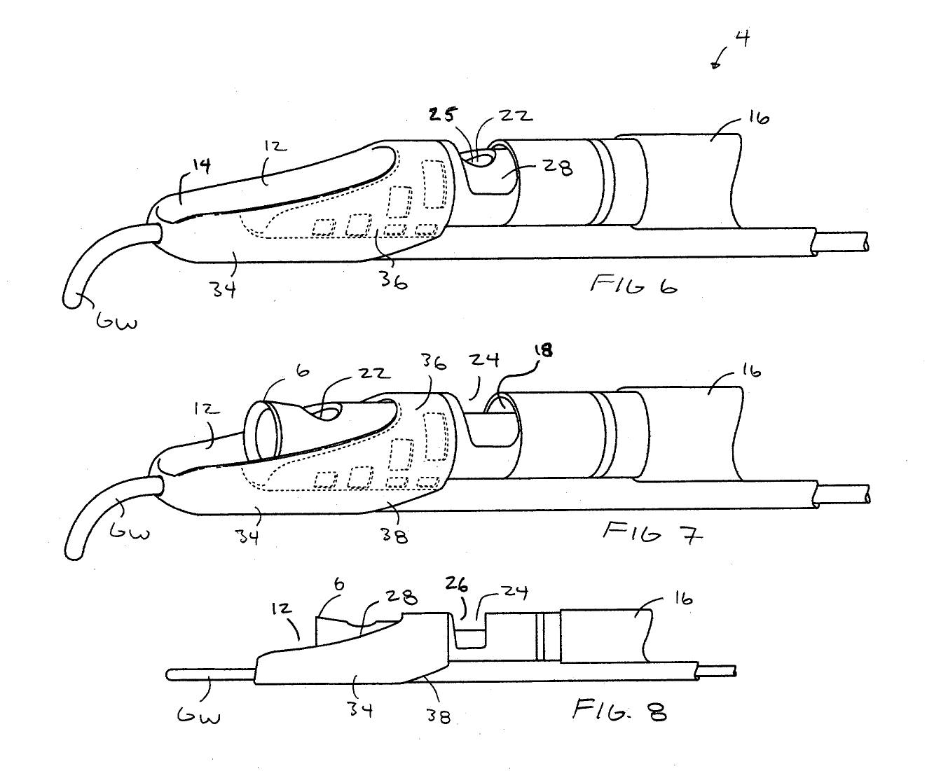

Referring to FIGS. 1-8, a system 2 and device 4 for reentering a true lumen from a subintimal space, dissection plane or so-called false lumen is shown. The device 4 includes a cutting element 6 coupled to a torque transmitting element 8, such as a wire 10, which rotates the cutting element 6. The device 4 has an opening 12 at a distal end 14 with the cutting element 6 movable between a stored position (FIG. 6) and a cutting position (FIGS. 7 and 8) which exposes the cutting element 6. The cutting element 6 may be any suitable cutting element 6 such as the cutting element 6 described in patents incorporated by reference above. The cutting element 6 has a circular cutting edge which has a diameter of about 1 mm although any suitable size may be used depending upon the particular application. The cutting element 6 may also be any other type of cutter such as a laser, ultrasound, RF or other type of cutter without departing from various aspects of the present invention.

The device 4 has a flexible body 16 to navigate through blood vessels or other body lumens to a target location. The body 16 may be made of any suitable material as is known in the art such as Pebax. The torque transmitting element 8 extends through a lumen 18 in the body 16. The body 16 may have more lumens for various reasons such as introduction of fluids, such as contrast, or for delivery of another device 4 such as a guidewire or interventional device. The torque transmitting element 8 is coupled to a driver 20 which rotates the torque transmitting element 8 at a variable or fixed speed.

The device 4 may also have an energy emitting element 22, such as an ultrasound element 25, which emits (and may receive) energy to determine the location of the true lumen as explained below. The energy emitting element 22 is coupled to the cutting element 6 so that the energy emitting element 22 and cutting element 6 are rotated together. The cutting element 6 is in the stored position when locating the true lumen so that the cutting element 6 is not exposed and will not cut or damage tissue. The energy emitting element 22 is positioned adjacent a window 24 which may be a side opening 26 or may simply be a portion of the sidewall which transmits a sufficient amount of the energy therethrough. Any suitable energy emitting element 22 may be used such as the ultrasound emitting element available from Boston Scientific and is marketed under the name Atlantis™. The cutting element 6 may be mounted to a collar which is then mounted to an ultrasound element holder 28 or the cutting element 6 may be integrally formed with the ultrasound element holder 28.

The device 4 has an atraumatic tip 34 which is relatively flexible to prevent damaging tissue. The tip 34 may be a separate piece laminated or glued to the body 16. The tip 34 is preferably made out of a relatively soft, flexible material, such as tecothane, and may be used for blunt dissection as necessary. A reinforcing element 36 is encapsulated in the tip 34 to help the tip 34 maintain it's general shape. The tip 34 may also have one or more guidewire lumens 38 or any of the guidewire features described herein.

The opening 12 in the distal portion may be designed to expose over 180 degrees of the cutting element 6 and may even expose 220 degrees or even 270 degrees of the cutting element 6 as defined by the axis of rotation. This provides advantages over WO 02/45598 which does not expose much of the cutting element 6 and requires invagination of the tissue within the opening to cut tissue. In another aspect of the invention, the cutting element 6 may be gradually exposed. For example, the cutting element 6 may be gradually exposed from 180-220 degrees or even 200-270 degrees. As explained below, this feature provides the user with the ability to change the amount of cutter 6 that is exposed depending upon the tissue thickness between the subintimal location and true lumen. The term opening 12 and amount of exposure of the cutting element 6 are defined by the outer bounds of the opening 12 and the axis of rotation. Referring to FIGS. 7 and 8, the cutting element 6 is exposed relative to the outer bounds of the opening 12 due to the relatively open distal end.

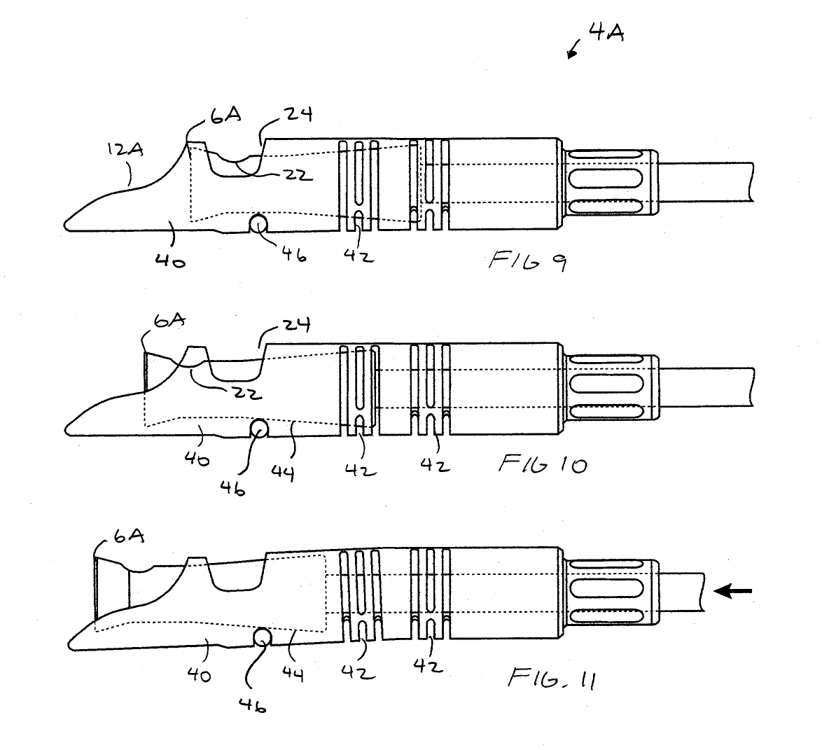

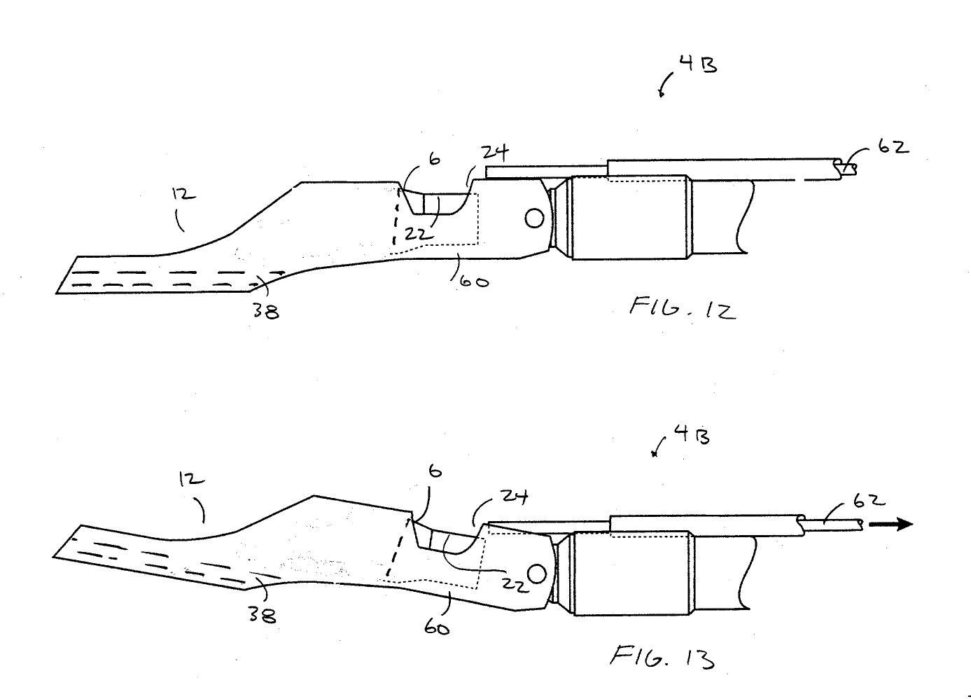

Referring to FIGS. 9-11, another device 4A for reentering a true lumen from a subintimal location is shown wherein the same or similar reference numbers refer to the same or similar structure. The device 4A also has an opening 12A at the distal end to expose the cutting element 6A. FIG. 9 shows the cutting element 6A in a stored position, FIG. 10 shows the cutting element 6A in a first cutting position and FIG. 11 shows the cutting element 6A in a second cutting position which further exposes the element 6A. The device 4A also has the window 24 through which the energy emitting element 22, such as the ultrasound element, may emit energy when the cutting element 6A is in the stored position.

A distal portion 40 of the body can bend or articulate to further expose the cutting element 6A and to move the cutting element 6A toward true lumen. The body has slots 42 formed therein to increase the flexibility of the distal portion 40. The cutting element 6A has a surface 44 which engages a lip 46 on the body. As the cutting element 6A is advanced, the interaction between the surface 44 and lip 46 causes the distal portion 40 to deflect. Bending the distal portion 40 can be helpful in moving the cutting element 6A toward the tissue and may also expose more of the cutting element 6A. As also explained below, the tip 40 may also be bent to direct the device 4A itself or a guidewire into the true lumen. The cutting element 6A may also be gradually exposed as the cutting element 6A moves distally and may be gradually exposed in the same manner described above.

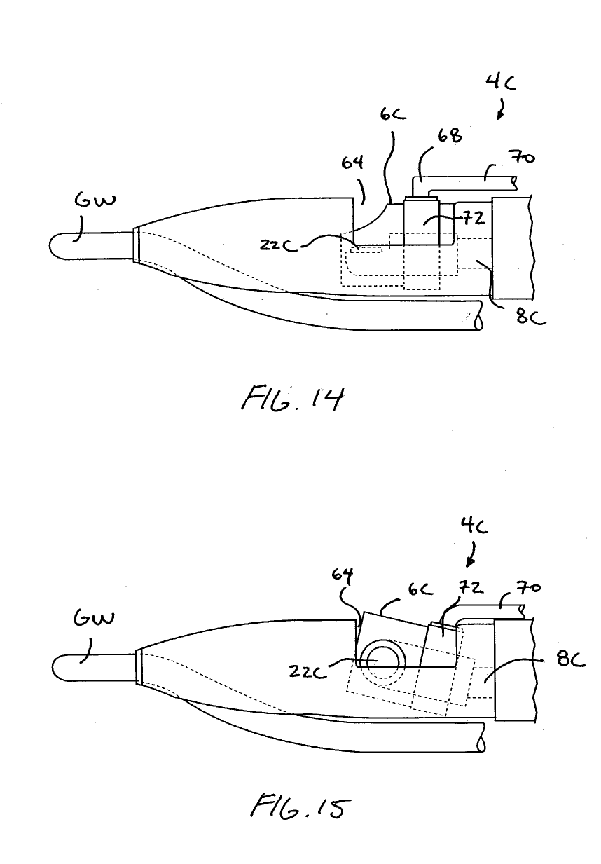

Referring to FIGS. 12 and 13, another reentry device 4B is shown which has a distal portion or tip 60 which bends or articulates. The tip 60 may be articulated and actuated in any suitable manner. For example, the tip 60 may be bent upon longitudinal movement of the cutting element 6 (as shown above) or a separate actuator, such as a pull wire 62, may be used. As can be appreciated from FIG. 13, the tip 60 is bent or articulated to move the cutting element 6 toward the true lumen and to expose more of the cutting element 6. The device 4B may also be bent to direct the device 4B itself or another device or guidewire through the guidewire lumen 38 to the access path into the true lumen as described further below.

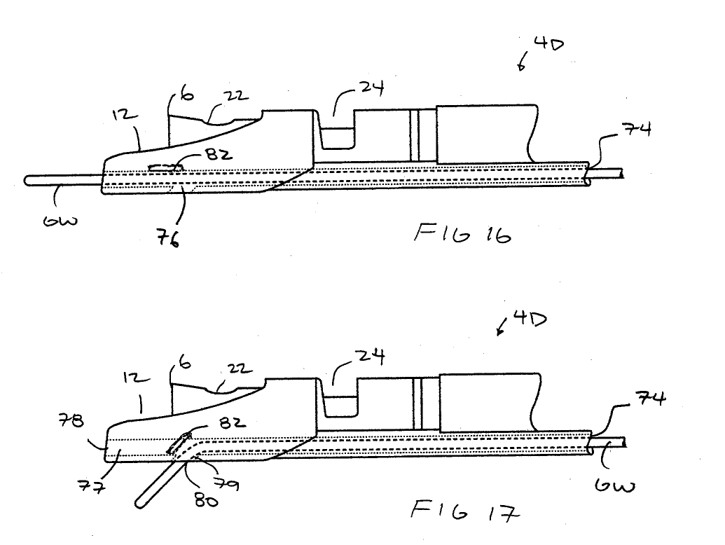

Referring to FIGS. 14 and 15, still another device 4C for cutting tissue is shown wherein the same or similar numbers refer to the same or similar structure. The device 4C includes a cutting element 6C, an energy emitting element 22C and a torque transmitter 8C for rotating the elements. The device 4C has an opening 64 along one side. The cutting element 6C is contained within the opening 64 in the stored position of FIG. 14 and extends out of the opening 64 in the cutting position of FIG. 15. The cutting element 6C is moved out of the window 24 using an actuator 68, such as a wire 70, which tilts a bearing 72 supporting the shaft of the rotatable cutting element 6C. Of course, any other suitable structure may be used to move the cutting element 6C outside the opening 64 such as those described in U.S. Pat. No. 6,447,525 which is hereby incorporated by reference. Furthermore, the cutting element 6C may be moved out of the opening 64 by bending the distal portion or tip as described herein.

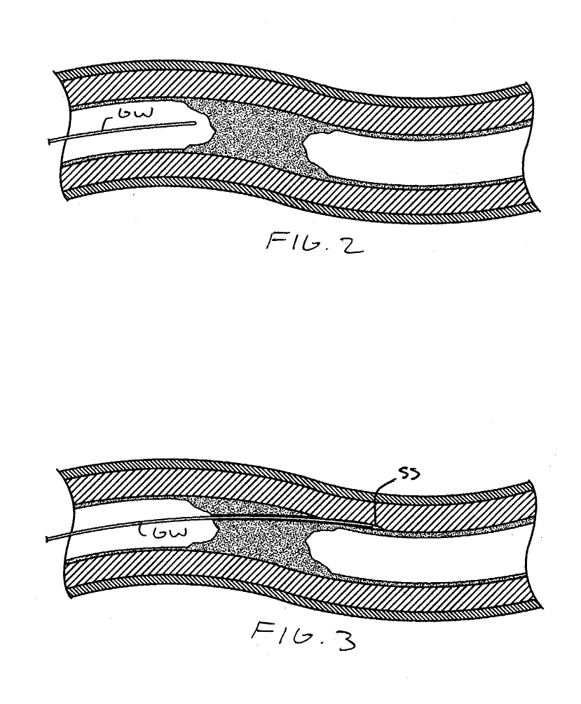

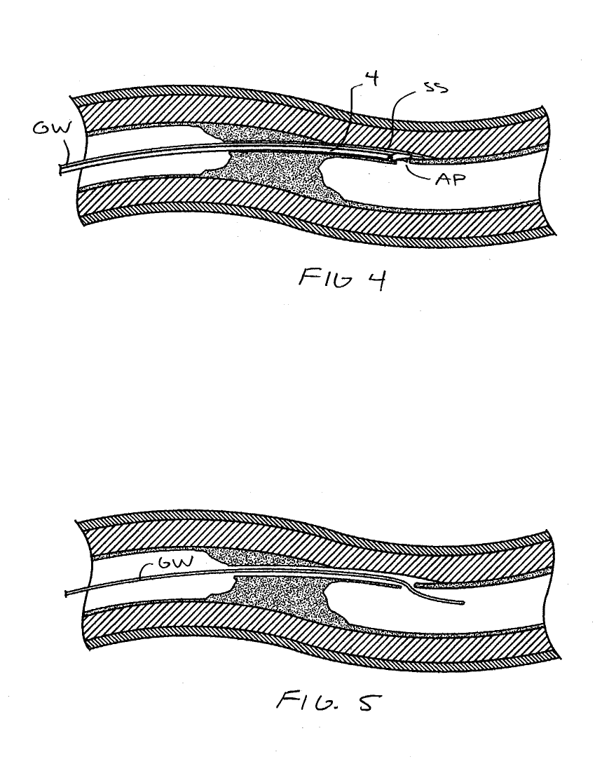

Use of the devices 4, 4A-C is now described with reference to the device 4 although it is understood that any of the devices 4, 4A-C may be used. As mentioned above, the device 4 may be used to perform any suitable procedure to cut from one location to another in the body such as a procedure to reenter a true lumen. The device 4 is initially advanced to a position within a subintimal space SS. As described above, the subintimal space SS may be inadvertently created during an endovascular procedure with a guidewire GW or other device creating the subintimal space SS as shown in FIGS. 2 and 3. The device 4 may be introduced over the same guidewire GW or device which created the subintimal space SS as shown in FIGS. 4 and 5. Of course, the device 4 may also be advanced over the guidewire GW to a position proximate to the subintimal space SS after which the device 4 is then advanced by itself into the subintimal space SS.

After the device 4 is positioned at the appropriate location in the subintimal space SS, the energy emitting element 22 is used to determine the location of the true lumen. When using the ultrasound element 28, for example, the ultrasound element 28 is rotated while emitting ultrasound energy and the energy emitted through the window 24 and reflected back through the window 24 is processed as is known in the art. The entire device 4 is rotated within the subintimal space SS to orient the window 24 until the true lumen is located. The angular orientation of the device 4 is then maintained so that the opening 12 and window 24 are directed toward the true lumen.

The cutting element 6 is then moved to the cutting position to expose the cutting element 6. The cutting element 6 may be rotated with the driver 20 during this time so that cutting is initiated as the cutting element 6 is exposed. In another aspect of the invention, the entire device 4 itself may be moved through the subintimal space to cut tissue. This provides advantages over the method of WO 02/45598 which requires invagination of tissue through a window to attempt a cut at one location. If the tissue does not invaginate sufficiently into the window, such as when the tissue is too thick, the device of WO 02/45598 will not be able to cut completely through the tissue to create the access path to the true lumen. The user must then move the device and again attempt to invaginate enough tissue to cut an access path. The present invention provides the ability to move the entire device 4 through the subintimal space to create the access path rather than attempting a cut at a single discrete location as in WO 02/45598. Of course, the device 4 may also be used by moving only the cutting element 6 rather than the entire device 4 without departing from the invention.

The cutting element 6 may also be exposed to varying degrees, as described above, until enough of the cutting element 6 is exposed to cut through to the true lumen. For example, the user may choose to expose half of the cutting element 6 and attempt to create an access path to the true lumen. If an access path is not created, the user may then choose to expose more of the cutting element 6 and again attempt to create an access path. This procedure can be repeated until the access path is formed to the true lumen. The device 4A, 4B may be also have a distal tip or portion 40, 60 which bends to move the cutting element 6 toward the tissue and/or expose more of the cutting element 6 during cutting.

After successfully creating the access path into the true lumen, the device 4 itself or part thereof may be directed toward or through the access path. Referring to FIG. 9-13, for example, the distal portion or tip 40, 60 may be bent to help direct the device 4A, 4B itself or the guidewire GW through the access path.

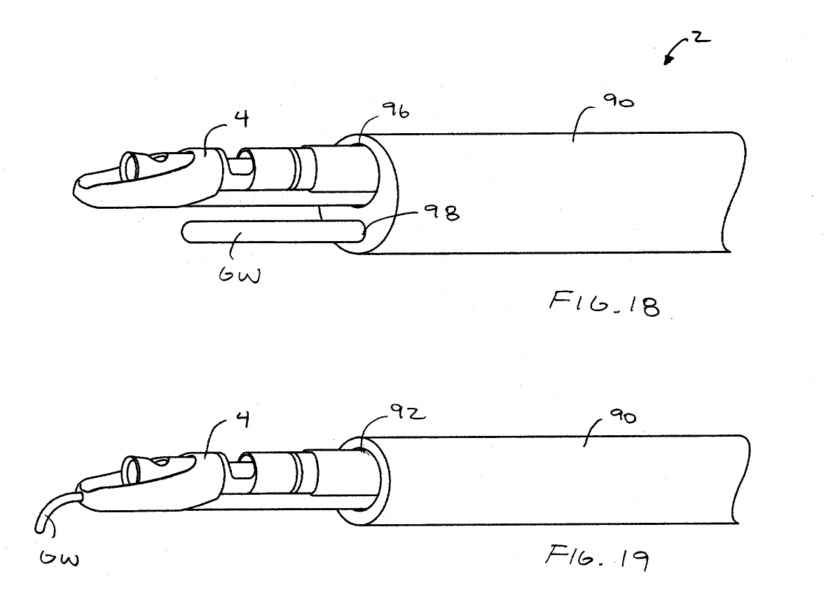

Referring to FIGS. 16 and 17, another device 4D, similar to device 4, is shown which has a guidewire lumen 74 having a junction 76 so the guidewire can be directed through either a first lumen 77 having a first outlet 78 or a second lumen 79 having a second outlet 80. The first outlet 78 directs the guidewire substantially longitudinally for advancing the device 4D over the guidewire to the target area in a conventional manner. The second outlet 80 directs the guidewire at an angle relative to the longitudinal axis, such as 30-75 degrees, to direct the guidewire through the access path into the true lumen.

The junction 76 may include a feature which directs the guidewire into the second outlet 80. Referring to FIG. 17, for example, the junction 76 may include a flap or stop 82 which closes and prevents or inhibits the guidewire from passing through the first outlet 78 after the guidewire has been withdrawn proximal to the junction 76. When the guidewire is advanced again as shown in FIG. 17, the guidewire passes through the second outlet 80 due to the stop 82. The device 4 and/or guidewire GW are then manipulated to direct the guidewire GW through the access path. Although the stop 82 may be provided, the junction 76 may also simply be a relatively open junction 76 with the user manipulating and rotating the guidewire GW to direct the guidewire GW through the desired outlet 78, 80. The device is rotated about 180 degrees after creating the access path to direct the GW through outlet 80 and into the true lumen.

Referring to FIGS. 18 and 19, the system 2 may also include a sheath or catheter 90 which is advanced proximal to the treatment site. The sheath 90 may help provide better control of the guidewire GW and devices 4 of the present invention during manipulation in the subintimal space. The sheath 90 may also used to deliver contrast solution to the treatment site from a source of contrast 97 (see FIG. 1) or may be coupled to a pressure sensor 94. The pressure sensor 94 may be part of the contrast delivery system 97 or may be a separate component. Deliver of contrast and/or pressure monitoring may be used to determine when the access path has been created.

The sheath 90 may include only one lumen 92 with fluid delivery and pressure sensing being accomplished in the annular space between the device and sheath as shown in FIG. 19. The sheath 90 may also have first and second lumens 96, 98 for separate delivery of the device 4 and guidewire GW. As mentioned above, the devices 4 of the present invention may be advanced over the same guidewire or device that created the subintimal space or may be advanced over another guidewire or even through the sheath 90 by itself.

After accessing the true lumen, another interventional device may be introduced into the true lumen for the intended therapy or procedure. For example, a stent catheter, angioplasty catheter, or atherectomy device may be used to treat the occlusion. The present invention has been described for reentering a true lumen from a subintimal space but, of course, may be used for other purposes to gain access from one space to another anywhere within the body.

The present invention has been described in connection with the preferred embodiments, however, it is understood that numerous alternatives and modifications can be made within the scope of the present invention as defined by the claims.

What is claimed is:

1. A method of re-entering a lumen during an endovascular procedure when in a subintimal space, comprising the steps of:
providing a device having a cutter, an opening, and an energy emitter coupled to the cutter, the cutter and energy emitter being coupled to a torque transmitting element which rotates the cutter and energy emitter together;
advancing the device through a lumen into a subintimal space adjacent to the lumen;
emitting energy from the energy emitter while in the subintimal space to locate the lumen after the advancing step;
moving the energy emitter and cutting element together to expose the cutting element to tissue; and
cutting through the wall using the rotating cutting element to provide an access path from the subintimal space to the lumen.

2. The method of claim 1, wherein:
the cutting step is carried out while moving the cutter and opening together through the subintimal space to create the access path into the true lumen.

3. The method of claim 1, wherein:
the providing step is carried out with the energy emitter being an ultrasound emitting element.

4. The method of claim 1, wherein:
the moving step is carried out to move at least part of the cutting element through the opening to expose the at least part of the cutting element.

5. The method of claim 1, further comprising the step of:
directing an element through the access path into the true lumen while maintaining the cutting element within the subintimal space

6. The method of claim 1, wherein:
the directing step is carried out with the element being a guidewire.

7. The method of claim 6, wherein:
the advancing step is carried out by advancing the device over the guidewire.

8. The method of claim 1, further comprising the step of:
directing an element through the device and through the access path into the true lumen.

9. The method of claim 1, wherein:
the providing step is carried out with the device having an open end;
the moving step being carried out with the cutting element moving from a position within the device to a position outside the open end to expose the cutting element.

10. The method of claim 1, wherein:
the moving step is carried out to move the cutting element to a position outside the opening.

11. The method of claim 1, wherein:
the moving step is carried out to expose over 180 degrees of the cutting element through the opening relative to an axis of rotation.

12. The method of claim 1, wherein:
the moving step is carried out to expose over 270 degrees of the cutting element relative to an axis of rotation.

13. The method of claim 1, wherein:
the moving step is carried out by gradually exposing more of the cutting element.

14. The method of claim 1, further comprising the step of:
bending a distal portion of the device before the cutting step.

15. The method of claim 14, wherein:
the bending step is carried out with an axis of rotation maintaining the same orientation relative to a proximal portion of the device.

16. The method of claim 1, wherein:
the cutting step is carried out while emitting energy with the energy emitting element.

17. The method of claim 1, further comprising the step of:
positioning a sheath proximal to the subintimal space.

18. The method of claim 17, further comprising the step of:
delivering a fluid through the sheath to determine whether the access path has been created.

19. The method of claim 17, further comprising:
advancing a guidewire through the sheath and through the access path into the true lumen.

20. A method of re-entering a lumen during an endovascular procedure, comprising the steps of:
providing a device having a rotatable cutter and an opening, the cutter being movable from a first position to a second position, the cutter being positioned within the opening in the first position and extending out of the opening in the second position;
advancing the device into a false lumen created during an endovascular procedure, the false lumen extending in a wall of a true lumen;
orienting the opening toward the true lumen after the advancing step;
moving the cutter into the second position so that the cutter extends out of the opening after the orienting step;
cutting an opening from the false lumen to the true lumen after the moving step; and
directing another endovascular device through the opening and into the true lumen.

21. The method of claim 20, wherein:
the orienting step is carried out using an energy emitting element.

22. The method of claim 21, wherein:
the orienting step is carried out with the energy emitting element being an ultrasound emitting element.

23. The method of claim 21, wherein:
the orienting step is carried out with the ultrasound emitting element rotating within a housing of the device.

24. The method of claim 20, wherein:
the providing step is carried out with the cutter being rotatable;
the cutting step is carried out while the cutter is rotating.

25. The method of claim 24, wherein:
the orienting step is carried out with the device having an ultrasound emitting element coupled to the cutter.

26. The method of claim 20, wherein:
the cutting step is carried out by moving the opening and the cutting element together within the false lumen.

27. The method of claim 26, wherein:
the cutting step is carried out by moving the entire device longitudinally to move the opening and the cutter relative to the wall of the true lumen.

28. The method of claim 20, further comprising the step of:
positioning a sheath proximal to the subintimal space.

29. The method of claim 28, further comprising the step of:
delivering a fluid through the sheath to determine whether the access path has been created.

30. The method of claim 28, further comprising:
advancing a guidewire through the sheath and through the access path into the true lumen.

31. The method of claim 20, wherein:
the moving step is carried out to expose over 180 degrees of the cutting element through the opening relative to an axis of rotation.

32. The method of claim 20, wherein:
the moving step is carried out to expose over 270 degrees of the cutting element relative to an axis of rotation.

33. The method of claim 20, wherein:
the moving step is carried out by gradually exposing more of the cutting element.

34. The method of claim 20, further comprising the step of:
bending a distal portion of the device before the cutting step.

35. The method of claim 34, wherein:
the bending step is carried out with an axis of rotation maintaining the same orientation relative to a proximal portion of the device.

36. A method of re-entering a lumen during an endovascular procedure, comprising the steps of:
providing a device having a cutter, a distal portion and a proximal portion, the distal portion being movable from a first condition to a second condition, the distal portion being angled relative to the proximal portion in the second condition;
advancing the device into a false lumen created during an endovascular procedure when the device is in the straightened condition;
moving the distal portion to the second condition to angle the distal portion after the advancing step;
orienting the opening toward a true lumen after the advancing step;
cutting an opening from the false lumen to the true lumen using the cutter; and
directing an endovascular device through the opening and into the true lumen.

37. The method of claim 36, wherein:
the directing step is carried out by directing the distal end of the device through the opening when in the second condition and then directing the endovascular device through the opening, the device being angled so that the distal portion is directed toward the access path cut during the cutting step.

38. The method of claim 36, wherein:
the directing step is carried out with the endovascular device being advanced over the device which creates the opening from the false lumen to the true lumen.

39. The method of claim 36, wherein:
the cutting step is carried out with the cutter rotating.

40. The method of claim 39, wherein:
the moving step is carried out with the cutter rotating around an axis of rotation, the axis of rotation maintaining the same orientation with respect to the proximal portion.

41. The method of claim 36, wherein:
the moving step is carried out by moving the cutter longitudinally which causes the distal portion to move to the second condition.

42. The method of claim 36, wherein:
the cutting step is carried out with the device being in the second condition; and
the directing step being carried out with the device also being in the second condition but with the device having an orientation which is about 180 degrees displaced from the orientation during the cutting step.

43. The method of claim 36, further comprising the step of:
positioning a sheath proximal to the subintimal space.

44. The method of claim 43, further comprising the step of:
delivering a fluid through the sheath to determine whether the access path has been created.

45. The method of claim 43, further comprising:
advancing a guidewire through the sheath and through the access path into the true lumen.

46. The method of claim 36, wherein:
the moving step is carried out to expose over 180 degrees of the cutting element through the opening relative to an axis of rotation.

47. The method of claim 36, wherein:
the moving step is carried out to expose over 270 degrees of the cutting element relative to an axis of rotation.

48. The method of claim 36, wherein:
the moving step is carried out by gradually exposing more of the cutting element.

49. The method of claim 36, further comprising the step of:
bending a distal portion of the device before the cutting step.

50. The method of claim 49, wherein:
the bending step is carried out with an axis of rotation maintaining the same orientation relative to a proximal portion of the device.

51. A method of cutting tissue in a blood vessel, comprising the steps of:
providing a device having a rotatable cutting element and an opening, the rotatable cutting element being movable from a stored position to a cutting position, the cutting element being exposed over at least 180 degrees in the cutting position;
advancing the device through a blood vessel;
moving the cutting element to the cutting position;
cutting tissue with the cutting element while the cutting element is rotating.

52. The method of claim 51, wherein:
the providing step is carried out with the device having a proximal portion and a distal portion, the proximal portion and the distal portion being movable relative to one another between a straight position to a bent position;
the cutting step being carried out with the device in the bent position.

53. The method of claim 52, wherein:
the cutting step is carried out with the distal portion bending relative to the proximal portion and an axis of rotation of the cutting element.

54. The method of claim 53, wherein:
the cutting step is carried out with the axis of rotation maintaining the same orientation with respect to the proximal portion when the device moves between the straight and bent positions.

55. The method of claim 51, wherein:
the providing step is carried out with the cutting element being exposed over at least 270 degrees.

56. The method of claim 51, further comprising the step of:
orienting the device so that the cutting element is exposed to cut tissue between a false lumen and a true lumen;
the advancing step being carried out with the device being positioned in the false lumen.

57. The method of claim 51, wherein:
the providing step is carried out with the device having an open end;
the moving step being carried out with the device moving through the open end into the cutting position.

58. The method of claim 51, wherein:
the providing step is carried out with the cutting element being gradually exposed from 220-240 degrees.

59. An endovascular tissue cutting device, comprising:
an elongate body having a lumen and an opening;
a torque transmitting element extending through the lumen; and
a rotatable cutter coupled to the torque transmitting element, the cutter being movable from a first position to a second position, the cutter being contained within the body in the first position and being exposed out of the opening in the second position, the rotatable cutter being exposed for over 180 degrees in the second position.

60. The device of claim 59, wherein:
the rotatable cutter is movable to a third position, the cutter being exposed for over 220 degrees in the third position.

61. The device of claim 59, wherein:
the rotatable cutter is movable to a third position, the cutter being exposed for over 270 degrees in the third position.

62. The device of claim 59, wherein:
the rotatable cutter has a gradually increasing exposure when moving toward the second position so that a user may change the amount of exposure of the cutter.

63. The device of claim 59, wherein:
the rotatable cutter gradually increases in exposure from 200-240 degrees when moving toward the second position.

64. A method of entering a true lumen from a subintimal location, comprising the steps of:
providing a device having an elongate body, a rotatable cutting element and an opening in the body, the rotatable cutting element being movable from a stored position to a cutting position, the cutting element being exposed through the opening when in the cutting position, the body being wider along a portion of the device where the cutting element is positioned in the cutting position to urge tissue toward the cutting element;
advancing the device through a blood vessel;
orienting the opening toward a true lumen;
moving the cutting element to the cutting position, wherein the wider portion of the body urges tissue toward the cutting element; and
cutting tissue with the cutting element while the cutting element is rotating.

65. The method of claim 64, wherein:
the providing step is carried out with the portion of the device where the cutting element is positioned in the cutting position exposes over 180 degrees of the cutting element.

66. The method of claim 65, wherein:
the providing step is carried out with the portion of the device where the cutting element is positioned in the cutting position exposes at least 270 degrees of the cutting element.

67. The method of claim 64, wherein:
the providing step is carried out with the device tapering distally after the wider portion, wherein the cutting element becomes increasingly exposed as the cutting element is moved distally.

68. The method of claim 64, wherein:
the providing step is carried out with the device having an open end which exposes substantially the entire cutting element when viewed along a longitudinal axis of the device.

69. The method of claim 64, further comprising the step of:
positioning a sheath proximal to the subintimal space.

70. The method of claim 69, further comprising the step of:
delivering a fluid through the sheath to determine whether the access path has been created.

71. The method of claim 69, further comprising:
advancing a guidewire through the sheath and through the access path into the true lumen.

72. A method of re-entering a lumen during an endovascular procedure, comprising the steps of:
providing a device having a rotatable cutter and an opening, the cutter being movable from a first position to a second position, the opening have a proximal side, the rotatable cutter being partially exposed at all positions distal to the proximal side after the cutter is initially exposed;
advancing the device into a false lumen created during an endovascular procedure, the false lumen extending through a wall of a true lumen, the cutter being positioned proximal to the proximal side of the opening;
orienting the opening toward the true lumen after the advancing step;
moving the cutter distally beyond the proximal side of the cutter so that the cutter extends out of the opening after the orienting step;
cutting an opening from the false lumen to the true lumen using the rotating cutter after the moving step; and
directing another endovascular device through the opening and into the true lumen.

73. The method of claim 72, wherein:
the providing step is carried out with the rotatable cutter being coupled to an energy emitting element which is coupled to the cutter; and
the orienting step being carried out by emitting energy from the energy emitting element to orient the opening.

74. The method of claim 73, wherein:
the moving step is carried out while emitting energy from the energy emitting element.

75. The method of claim 72, wherein:
the cutting step is carried out by moving the entire device longitudinally so that the rotating cutter translates within the false lumen to cut through the wall to the true lumen.

76. The method of claim 72, wherein:
the providing step is carried out with the device having a lumen in communication with the opening.

77. The method of claim 76, further comprising the step of:
introducing a fluid through the lumen.

78. The method of claim 76, wherein:
the introducing step is carried out with the fluid being contrast.

79. The method of claim 76, further comprising the step of:
measuring a pressure through the lumen.

80. The method of claim 72, further comprising the step of:
positioning a sheath proximal to the subintimal space.

81. The method of claim A80, further comprising the step of:
delivering a fluid through the sheath to determine whether the access path has been created.

82. The method of claim 80, further comprising:
advancing a guidewire through the sheath and through the access path into the true lumen.

83. The method of claim 72, wherein:
the moving step is carried out to expose over 180 degrees of the cutting element through the opening relative to an axis of rotation.

84. The method of claim 72, wherein:
the moving step is carried out to expose over 270 degrees of the cutting element relative to an axis of rotation.

85. The method of claim 72, wherein:
the moving step is carried out by gradually exposing more of the cutting element.

86. The method of claim 72, further comprising the step of:
bending a distal portion of the device before the cutting step.

87. The method of claim 86, wherein:
the bending step is carried out with an axis of rotation maintaining the same orientation relative to a proximal portion of the device.

88. A method of re-entering a lumen during an endovascular procedure, comprising the steps of:
providing a device having a rotatable cutter and an opening, the cutter being movable relative to the opening between a first position and a second position, the cutter being increasingly exposed when moving from the first position to the second position;
advancing the device into a false lumen created during an endovascular procedure, the false lumen extending in a wall of a true lumen;
orienting the opening toward the true lumen after the advancing step;
moving the cutter from the first position toward the second position to expose part of the cutter;
determining whether an access path has been created from the false lumen to the true lumen;
increasing the amount of the cutter that is exposed until the access path has been created; and
directing another endovascular device through the opening and into the true lumen.

89. The method of claim 88, wherein:
the providing step is carried out with the cutter being exposed for over 180 degrees when in the second position.

90. The method of claim 89, wherein:
the providing step is carried out with the cutter being exposed for over 220 degrees when in the second position.

91. The method of claim 88, wherein:
the providing step is carried out with the cutter being having a gradually increasing exposure when moving toward the second position.

92. The method of claim 91, wherein:
the providing step is carried out with the cutter being increasing exposed from at least 180 degrees to at least 220 degrees when moving from the first position to the second position.

93. The method of claim 88, further comprising the step of:
positioning a sheath proximal to the subintimal space.

94. The method of claim 93, further comprising the step of:
delivering a fluid through the sheath to determine whether the access path has been created.

95. The method of claim 93, further comprising:
advancing a guidewire through the sheath and through the access path into the true lumen.

96. An intravascular incising device, comprising:
an elongate body, the body having at least one lumen and being sufficiently flexible to be advanced through a patient's vasculature to a treatment site;
a torque transmitting element extending through the lumen;
a cutting element coupled to the torque transmitting element, the cutting element being movable relative to the opening to a number of discrete positions to vary an amount of the cutting element which is exposed relative to the opening.

97. The device of claim 96, wherein:
the cutting element is movable from a stored position in which the cutting element is not exposed to a working position where the cutting element is exposed at least 180 degrees relative to the opening.

98. The device of claim 96, wherein:
the cutting element is movable from a stored position in which the cutting element is not exposed to a working position where the cutting element is exposed at least 180 degrees relative to the opening.

99. The device of claim 96, wherein:
the cutting element is movable from a stored position in which the cutting element is not exposed to a working position where the cutting element is exposed at least 270 degrees relative to the opening.

100. The device of claim 96, wherein:
the cutting element may be gradually exposed between 180-220 degrees.

101. The device of claim 96, wherein:
the cutting element may be gradually exposed between 200-270 degrees.

102. The device of claim 96, wherein:
the cutting element may be moved longitudinally relative to the body for moving the cutting element between the number of discrete positions.

103. A method of entering a true lumen from a false lumen during an endovascular procedure, comprising the steps of:
providing a guidewire and a reentry device;
positioning the guidewire at a subintimal space;
advancing the reentry device over the guidewire to the target location;
cutting an access path from the subintimal space to a true lumen; and
directing the guidewire through the access path into the true lumen.

104. The method of claim 103, further comprising the step of:
advancing an endovascular device over the guidewire and through the access path.

105. The method of claim 103, wherein:
the directing step is carried out by directing the guidewire through a different distal opening than during the advancing step.

106. The method of claim 105, wherein:
the directing step is carried out with the device having a junction with a first arm and a second arm;
the advancing step being carried out with the guidewire passing through the first arm; and
the directing step is carried out with the guidewire passing through the second arm of the junction.

107. The method of claim 103, wherein:
the directing step is carried out with a distal portion of the device being bent.

108. The method of claim 103, wherein:
the directing step is carried out with the reentry device extending through the access path, the guidewire being advanced through the reentry device while the reentry device is positioned in the access path.

109. The method of claim 103, further comprising the step of:
positioning a sheath proximal to the subintimal space.

110. The method of claim 109, further comprising the step of:
delivering a fluid through the sheath to determine whether the access path has been created.

111. The method of claim 109, further comprising:
advancing another guidewire through the sheath and through the access path into the true lumen.

112. The method of claim 1, further comprising the step of:
measuring a pressure to determine when the access path has been created.

113. The method of claim 20, further comprising the step of:
measuring a pressure to determine when an access path has been created.

114. The method of claim 36, further comprising the step of:
measuring a pressure to determine when the opening into the true lumen has been created.

115. The method of claim 88, further comprising the step of:
measuring a pressure to determine when the access path has been created.

116. The method of claim 103, further comprising the step of:
measuring a pressure to determine when the access path has been created.
